(19) United States
(12) Patent Application Publication
(10) Pub. No.: US 2004/0175359 A1
(43) Pub. Date: Sep. 9, 2004

(54) Novel proteins with antiviral, antineoplastic, and/or immunomodulatory activity

(76) Inventors: John Rudolph Desjarlais, Pasadena, CA (US); Shannon Alicia Marshall, San Francisco, CA (US); Yirong Mo, Kalamazoo, MI (US); Adam Read Thomason, Pasadena, CA (US)

Correspondence Address: Robin M. Silva, DORSEY & WHITNEY LLP, Suite 3400, Four Embarcadero Center, San Francisco, CA, 94111-4187, US

(21) Appl. No.: 10/677,093

(22) Filed: Sep. 30, 2003

Related U.S. Application Data
(60) Provisional application No. 60/425,851, filed on Nov. 12, 2002.

(51) Int. Cl. A61K 38/21; C07H 21/04; C12P 21/04; C07K 14/555

(52) U.S. Cl. 424/85.4; 424/85.5; 424/85.6; 530/351; 435/69.51; 435/320.1; 435/325; 536/23.5

(57) ABSTRACT

The invention relates to interferon variants with improved properties and methods for their use.

This application claims benefit of priority under 35 USC 119(e)(1) to U.S. S No.: 60/425,851 hereby incorporated by reference in its entirety.

### FIELD OF THE INVENTION

This invention relates involves the use of circular permutation or cyclization to create novel proteins with properties related to a known protein activity.

### BACKGROUND OF THE INVENTION

Interferons (IFNs) are a well-known family of cytokines possessing a range of biological activities including antiviral, anti-proliferative, and immunomodulatory activities. Interferons have demonstrated utility in the treatment of a variety of diseases, and are in widespread use for the treatment of multiple sclerosis and viral hepatitis.

Interferons (IFNs) are a well-known family of cytokines; they may be classified into groups by their chemical and biological characteristics. Interferons include a number of related proteins, such as interferon-alpha (IFN-α), interferon-beta (IFN-β), interferon-gamma (IFN-γ) interferon-kappa (IFN-κ, also known as interferon-epsilon or IFN-ε), interferon-tau (IFN-τ), and interferon-omega (IFN-ω). These interferon proteins are produced in a variety of cell types: IFN-α (leukocytes), IFN-β (fibroblasts), IFN-γ (lymphocytes), IFN-ε or κ (keratinocytes), IFN-ω (leukocytes) and IFN-τ (trophoblasts). IFN-α, IFN-β, IFN-ε or κ, IFN-ω, and IFN-τ are classified as type I interferons, while IFN-γ is classified as a type II interferon. Interferon alpha is encoded by a multi-gene family, while the other interferons appear to each be coded by a single gene in the human genome. Furthermore, there is some allelic variation in interferon sequences among different members of the human population.

Type-I interferons all appear to bind a common receptor, type I IFN-R, composed of IFNAR1 and IFNAR2 subunits. The exact binding mode and downstream signal transduction cascades differ somewhat among the type I interferons. However, in general, the JAK/STAT signal transduction pathway is activated following binding of interferon to the interferon receptor. STAT transcription factors then translocate to the nucleus, leading to the expression of a number of proteins with antiviral, antineoplastic, and immunomodulatory activities.

Naturally occurring interferons possess antiviral, antiproliferative, and immunomodulatory activities, making interferons valuable therapeutics. However, drugs based on naturally occurring interferons suffer from a number of liabilities, including a high incidence of side effects and immunogenicity.

The present invention is directed to interferon proteins with improved properties. A number of groups have generated modified interferons with improved properties; the references below are all expressly incorporated by reference in their entirety.

Cysteine-depleted variants have been generated to minimize formation of unwanted inter- or intra-molecular disulfide bonds (U.S. Pat. No. 4,518,584; U.S. Pat. No. 4,588,585; U.S. Pat. No. 4,959,314). Methionine-depleted variants have been generated to minimize susceptibility to oxidation (EP 260350).

Interferons with modified activity have been generated (U.S. Pat. No. 6,514,729; U.S. Pat. No. 4,738,844; U.S. Pat. No. 4,738,845; U.S. Pat. No. 4,753,795; U.S. Pat. No. 4,766,106; WO 00/78266). U.S. Pat. Nos. 5,545,723 and 6,127,332 disclose substitution mutants of interferon beta at position 101. Chimeric interferons comprising sequences from one or more interferons have been made (Chang et. al. Nature Biotech. 17: 793-797 (1999), U.S. Pat. No. 4,758,428; U.S. Pat. No. 4,885,166; U.S. Pat. No. 5,382,657; U.S. Pat. No. 5,738,846). Substitution mutations to interferon beta at positions 49 and 51 have also been described (U.S. Pat. No. 6,531,122).

Interferons have been modified by the addition of polyethylene glycol (“PEG”) (see U.S. Pat. No. 4,917,888; U.S. Pat. No. 5,382,657; WO 99/55377; WO 02/09766; WO 02/3114). PEG addition can improve serum half-life and solubility. Serum half-life can also be extended by complexing with monoclonal antibodies (U.S. Pat. No. 5,055,289), by adding glycosylation sites (EP 529300), by co-administering the interferon receptor (U.S. Pat. No. 6,372,207), by preparing single-chain multimers (WO 02/36626) or by preparing fusion proteins comprising an interferon and an immunoglobulin or other protein (WO 01/03737, WO 02/3472, WO 02/36628).

Interferon alpha and interferon beta variants with reduced immunogenicity have been claimed (See WO 02/085941 and WO 02/074783). Due to the large number of variants disclosed and the apparent lack of structural and functional effects of the introduced mutations, identifying a variant that would be a functional, less immunogenic interferon variant suitable for administration to patients may be difficult.

Interferon variants with improved solubility and soluble expression have been generated (See U.S. Ser. No.______, filed Sep. 29, 2003, titled Interferon Variants With Improved Properties, incorporated by reference in its entirety herein). Interferon beta variants with enhanced stability have also been claimed, in which the hydrophobic core was optimized using rational design methods (WO 00/68387). Alternate formulations that promote interferon stability or solubility have also been disclosed (U.S. Pat. No. 4,675,483; U.S. Pat. No. 5,730,969; U.S. Pat. No. 5,766,582; WO 02/38170). Interferon beta muteins with enhanced solubility have been claimed, in which several leucine and phenylalanine residues are replaced with serine, threonine, or tyrosine residues (WO 98/48018).

There exists a need for the development and discovery of interferon proteins with improved properties, including but not limited to increased efficacy, decreased side effects, decreased immunogenicity, increased solubility, suitability for non-injection based modes of administration, and enhanced soluble prokaryotic expression. Improved interferon therapeutics may be useful for the treatment of a variety of diseases and conditions, including autoimmune diseases, viral infections, and inflammatory diseases, cancer, among others. In addition, interferons may be used to promote the establishment of pregnancy in certain mammals.

Cyclic or circularly permuted interferon variants may exhibit improved protein properties relative to the naturally occurring interferon proteins. Interferons, like all natural proteins, have an amino acid sequence beginning with an N-terminus and ending with a C-terminus. The N- and C-termini may be joined to create a cyclized protein. A circularly permuted protein may then be generated by creating new N- and C-termini between a pair of residues that are located internally in the naturally occurring sequence.

In some cases, naturally occurring pairs of proteins have been identified that are related by linear reorganization of their amino acid sequences. Such sequences can be considered to be naturally occurring circularly permuted proteins. (see for example Cunningham, et al., Proc. Natl. Acad. Sci. U.S.A. 76:3218-3222, 1979; Teather & Erfle, J. Bacteriol. 172: 3837-3841, 1990; Schimming et al., Eur. J. Biochem. 204: 13-19, 1992; Yamiuchi and Minamikawa, FEBS Lett. 260:127-130, 1991: MacGregor et al., FEBS Lett. 378:263-266, 1996).

Circular permutants of proteins and cyclic proteins may have improved or altered physical, chemical, and/or biological properties such as enhanced stability, solubility, and activity or altered immunogenicity or pharmacokinetics as compared to the wild-type protein (see for example Sanders et. al., Blood 100: 299-305 (2002) and Osuna et. al. Prot. Eng. 15: 463-470 (2002)).

Accordingly, it is an object of the present invention to provide circular permutants of IFN proteins with desired properties.

### SUMMARY OF THE INVENTION

The invention provides for the use of cyclization and circular permutation technologies to create novel proteins with desired physical, chemical, and/or biological properties. The invention also provides methods for the production of novel proteins that have similar biological activity to existing proteins. The invention further provides methods for the production of novel proteins that have physical, chemical, and/or biological properties that differ from the wild type protein. For example, the novel proteins may possess enhanced stability, solubility, or activity or altered immunogenicity or pharmacokinetics as compared to the wild-type protein.

It is an object of the present invention to provide novel proteins with increased stability and/or solubility with antiviral, antineoplastic, and/or immunomodulatory activity, including but not limited to modified interferons (IFNs).

It is a further object of the present invention to provide altered pharmacokinetics and/or altered immunogenicity of a novel protein with antiviral, antineoplastic, and/or immunomodulatory activity, including but not limited to modified IFNs.

### BRIEF DESCRIPTION OF THE DRAWINGS

FIG. 1 shows amino acid sequences for type I interferons.

FIG. 2 shows a sequence alignment of human interferon-alpha subtypes.

FIG. 3 shows a sequence alignment of IFN-alpha 2a (1 ITF), IFN-beta (1AU1), IFN-kappa (IFNK), and IFN-tau (1B5L).

FIG. 4. shows (a) the structure of wild type IFN-a2a obtained from PDB code 1ITF, and (b) the structure of a circularly permuted variant of IFN-a2a.

FIG. 5. shows (a) the structure of wild type IFN-β obtained from PDB code 1AU1, and (b) the structure of a circularly permuted variant of IFN-β.

### DETAILED DESCRIPTION OF THE INVENTION

By “control sequences”—and grammatical equivalents herein is meant nucleic acid sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. By “interferon-responsive disorders” and grammatical equivalents herein is meant diseases, disorders, and conditions that can benefit from treatment with a type I interferon. Examples of interferon-responsive disorders include, but are not limited to, autoimmune diseases (e.g. multiple sclerosis, diabetes mellitus, lupus erythematosus, Crohn's disease, rheumatoid arthritis, stomatitis, asthma, allergies and psoriasis), viral infections (e.g. hepatitis C, papilloma viruses, hepatitis B, herpes viruses, viral encephalitis, cytomegalovirus, and rhinovirus), and cell proliferation diseases or cancer (e.g. osteosarcoma, basal cell carcinoma, cervical dysplasia, glioma, acute myeloid leukemia, multiple myeloma, chronic lymphocytic leukemia, Kaposi's sarcoma, chronic myelogenous leukemia, renal-cell carcinoma, ovarian cancers, hairy-cell leukemia, and Hodgkin's disease). Interferons may also be used to promote the establishment of pregnancy in certain mammals. By “modification” and grammatical equivalents is meant insertions, deletions, or substitutions to a protein or nucleic acid sequence. Circularly permutation and cyclization are also included in the definition of modification. By “naturally occurring” or “wild type” or “wt” and grammatical equivalents thereof herein is meant an amino acid sequence or a nucleotide sequence that is found in nature and includes allelic variations. In a preferred embodiment, the wild-type sequence is the most prevalent human sequence. However, the wild type IFN proteins may be from any number of organisms, include, but are not limited to, rodents (rats, mice, hamsters, guinea pigs, etc.), primates, and farm animals (including sheep, goats, pigs, cows, horses, etc). By “nucleic acid” and grammatical equivalents herein is meant DNA, RNA, or molecules, which contain both deoxy- and ribonucleotides. Nucleic acids include genomic DNA, cDNA and oligonucleotides including sense and anti-sense nucleic acids. Nucleic acids may also contain modifications, such as modifications in the ribose-phosphate backbone that confer increased stability and half-life. Nucleic acids are “operably linked” when placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading frame. However, elements such as enhancers do not have to be contiguous. A “patient” for the purposes of the present invention includes both humans and other animals, particularly mammals, and organisms. Thus the methods are applicable to both human therapy and veterinary applications. In the preferred embodiment the patient is a mammal, and in the most preferred embodiment the patient is human. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. By “protein” herein is meant a molecule comprising at least two covalently attached amino acids, which includes proteins, polypeptides, oligopeptides and peptides. The protein may be made up of naturally occurring amino acids and peptide bonds, or synthetic peptidomimetic structures such as peptoids (see Simon et al., Proc. Natl. Acad. Sci. U.S.A. 89(20:9367-71 (1992)). For example, homo-phenylalanine, citrulline, and noreleucine are considered amino acids for the purposes of the invention, and both D- and L-amino acids may be utilized. By “protein properties” herein is meant biological, chemical, and physical properties including but not limited to enzymatic activity, specificity (including substrate specificity, kinetic association and dissociation rates, reaction mechanism, and pH profile), stability (including thermal stability, stability as a function of pH or solution conditions, resistance or susceptibility to ubiquitination or proteolytic degradation), solubility, aggregation, structural integrity, crystallizability, binding affinity and specificity (to one or more molecules including proteins, nucleic acids, polysaccharides, lipids, and small molecules), oligomerization state, dynamic properties (including conformational changes, allostery, correlated motions, flexibility, rigidity, folding rate), subcellular localization, ability to be secreted, ability to be displayed on the surface of a cell, posttranslational modification (including N- or C-linked glycosylation, lipidation, and phosphorylation), amenability to synthetic modification (including PEGylation, attachment to other molecules or surfaces), and ability to induce altered phenotype or changed physiology (including cytotoxic activity, immunogenicity, toxicity, ability to signal, ability to stimulate or inhibit cell proliferation, ability to induce apoptosis, and ability to treat disease). When a biological activity is the property, modulation in this context includes both an increase or a decrease in activity. By “therapeutically effective dose” herein is meant a dose that produces the effects for which it is administered. The exact dose will depend on the purpose of the treatment, and will be ascertainable by one skilled in the art using known techniques. As is known in the art, adjustments for variant IFN protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art. By “treatment” herein is meant to include therapeutic treatment, as well as prophylactic, or suppressive measures for the disease or disorder. Thus, for example, successful administration of a variant IFN protein prior to onset of the disease may result in treatment of the disease. As another example, successful administration of a variant IFN protein after clinical manifestation of the disease to combat the symptoms of the disease comprises “treatment” of the disease. “Treatment” also encompasses administration of a variant IFN protein after the appearance of the disease in order to eradicate the disease. Successful administration of an agent after onset and after clinical symptoms have developed, with possible abatement of clinical symptoms and perhaps amelioration of the disease, further comprises “treatment” of the disease. By “variant interferon nucleic acids” and grammatical equivalents herein is meant nucleic acids that encode variant interferon proteins. Due to the degeneracy of the genetic code, an extremely large number of nucleic acids may be made, all of which encode the variant interferon proteins of the present invention, by simply modifying the sequence of one or more codons in a way that does not change the amino acid sequence of the variant interferon. By “variant interferon proteins” or “non-naturally occurring interferon proteins” and grammatical equivalents thereof herein is meant non-naturally occurring interferon proteins which differ from the wild type interferon protein by at least one (1) amino acid insertion, deletion, or substitution, or by circular permutation or cyclization. Interferon variants are characterized by the predetermined nature of the variation, a feature that sets them apart from naturally occurring allelic or interspecies variation of the interferon protein sequence. The cyclized or circularly permuted variant interferon proteins may additionally contain insertions, deletions, and/or substitutions at the N-terminus, C-terminus, or internally, for instance mutations that alter additional protein properties such as stability or immunogenicity or which enable or prevent posttranslational modifications such as PEGylation or glycosylation. Variant interferon proteins may be subjected to co- or post-translational modifications, including but not limited to synthetic derivatization of one or more side chains or termini, glycosylation, PEGylation, fusion to proteins or protein domains, and addition of peptide tags or labels.

Interferons, like all natural proteins, have an amino acid sequence beginning with an N-terminus and ending with a C-terminus. The N- and C-termini may be joined to create a cyclized protein. A circularly permuted protein may be generated by creating new N- and C-termini between a pair of residues (the “breakpoint”) that are located internally in the naturally occurring sequence. As a result, the sequence of the circular permutant comprises (1) the sequence of the original protein from the breakpoint to the C-terminus, and (2) the sequence of the original protein from the N-terminus to the breakpoint. The sequence may additionally comprise a “linker” of one or more residues located between the original C-terminus and the original N-terminus. Furthermore, the sequence of the circular permutant may be further altered relative to the original protein, especially in the region of the original termini or breakpoint.

Note that the circularly permuted interferon proteins, when aligned globally, share little to no sequence similarity with wild type interferon sequences.

As is known in the art, cyclization and circular permutation can be applied to any protein, but are best suited to proteins wherein the N- and C-termini are located close in space in the 3-dimensional structure of the protein. Type one interferons, including but not limited to interferon-alpha, interferon-beta, interferon-kappa, interferon-tau, and interferon-omega, are structurally well-suited to cyclization and circular permutation.

Various techniques may be used to permutate proteins. See U.S. Pat. No. 5,981,200; Maki K, Iwakura M., Seikagaku. 2001 January; 73(1): 42-6; Pan T., Methods Enzymol. 2000; 317:313-30; Heinemann U, Hahn M., Prog Biophys Mol Biol. 1995; 64(2-3): 121-43; Harris M E, Pace N R, Mol Biol Rep. 1995-96; 22(2-3):115-23; Pan T, Uhlenbeck O C., 1993 Mar. 30; 125(2): 111-4; Nardulli A M, Shapiro D J. 1993 Winter; 3(4):247-55, EP 1098257 A2; WO 02/22149; WO 01/51629; WO 99/51632; Hennecke, et al., 1999, J. Mol. Biol., 286, 1197-1215; Goldenberg et al J. Mol. Biol 165, 407-413 (1983); Luger et al, Science, 243, 206-210 (1989); and Zhang et al., Protein Sci 5, 1290-1300 (1996); all hereby incorporated by reference.

Methods for Generating Cyclic and Circularly Permuted Proteins

In a preferred embodiment, cyclic proteins are generated utilizing INTEIN technology. Thus, peptides can be cyclized and in particular inteins may be utilized to accomplish the cyclization. In an alternate embodiment, other techniques include making chimeric peptides. See, WO 00/36903; Iwakura et al, Nature Structural Biology, Vol 7, No. 7, pages580-585 and references cited therein (2000): Henneke et al, J. Mol Biol (1999) 286, 1197-1215 and all references cited therein (1999); Goldenberg et al J. Mol. Biol 165, 407-413 (1983); Luger et al, Science, 243, 206-210 (1989); Zhang et al., Protein Sci 5, 1290-1300 (1996); Holford et al, Structure, vol. 6, 15 Aug. 1998, pages 951-956; Southworth et al., EMBO Journal, GB Oxford University Press, vol 17, No. 4, 1998, pages 918-926; WO 97 01642 A; Scott et al, Proceedings of the National Academy of Sciences of US, Vol. 96, No. 24 pages 13638-13643 (Nov. 23, 1999); Evans et al., J. of Biological Chemistry, Vol 274, No. 26, 18359-18363 (1999); Iwai et al, FEBS Letters, vol 459, No. 2, pages 166-172 (1999); U.S. Pat. No. 6,365,377; U.S. Pat. No. 5,795,931; and WO 00047751 A1; EP 0759944 B1; WO 95/31483; WO0034317A2 and A3; WO 98/33523 A1; WO 00136624 A1; WO 9852976 A1; WO 9911777 A1; hereby incorporated by reference. Any of these techniques may be used to generate the proteins of the present invention.

Selection of Suitable Locations for the New Termini

In a preferred embodiment, the novel N- and C-termini are located outside of regular secondary structural elements, e.g. the novel termini are located in a loop or turn, such that the stability and activity of the novel protein are similar to those of the original protein.

In another preferred embodiment, the breakpoint is selected to alter one or more properties of the protein. For example, if a protein of interest is prone to unwanted proteolytic cleavage at a particular site, that site may be selected as the breakpoint such that the resulting circularly permuted protein does not contain the unwanted cleavage site. Preferred breakpoints may include glycosylation sites, the binding sites of non-neutralizing antibodies, or proteolytic cleavage sites. (See U.S. Pat. No. 6,100,070 and WO 98/18926). Similarly, the breakpoint may be selected to disrupt binding to a specific protein receptor.

Suitable locations for new termini in interferon-alpha include, but are not limited to, between residues 27 and 28; 48 and 49; 76 and 77; and 105 and 106. Other positions, particularly those close to the aforementioned positions (e.g. 101/103, 107/108, etc.), are also possible.

Suitable locations for new termini in interferon-beta include, but are not limited to, between residues 77 and 78; 27 and 28; 109 and 110; 136 and 137; and 47 and 48. Other positions, particularly those close to the aforementioned positions (e.g. 77 and 79; 75 and 76, etc.) are also possible.

Suitable locations for new termini in interferon-kappa include, but are not limited to, between residues 32 and 33; 48 and 49; 81 and 82; 118 and 119; and 148 and 149. Other positions, particularly those close to the aforementioned positions are also possible.

Selection of Appropriate Linker Sequences

In a preferred embodiment, the original N- and C-termini are joined via a peptide linker comprising from 0 to 30 amino acids. Appropriate linker sequences may be obtained in a number of ways.

In one embodiment, the peptide linker joining the original N- and C-termini is a sequence of suitable length that is highly flexible. For instance, as is known in the art, linkers comprising one or more repeats of glycine-glycine-glycine-glycine-serine may be used.

In another alternate embodiment, the peptide linker joining the original N- and C-termini is obtained using de novo loop modeling following by selection of side chain identities for the loop residues.

In a preferred embodiment, the peptide linker joining the original N- and C-termini is a loop of suitable length obtained from a protein with local structural similarity to the original termini.

For example, suitable linkers for connecting residues 9 and 159 IFN-alpha include the residues R4129-A4130-G4131-N4132 obtained from PDB code 1LA0, W56-A57-S58-T59 obtained from chain L in PDB code 1A5F, C2019-G2020-N2021-K2022 obtained from chain B in PDB code 1DFC, and Q377-N378-T379-K380-S381 from chain B in PDB code 1D5S.

As another example, suitable linkers for connecting residues 4 and 165 in IFN-beta include the residues G74-D75 from PDB code 1HBG, the residues D213-T214 from PDB code 1EK4, and the residues Q575-S576 from chain B in PDB code 1E3A.

Additional Modifications

Additional insertions, deletions, and substitutions may be incorporated into the variant interferon proteins of the invention in order to confer other desired properties.

It is possible to add or remove one or more amino acids located at the original N- and/or C-termini in order to accommodate linker design. For example, residues may be removed to decrease the distance in space that the linker must span. Furthermore, one or more residues may be added or removed from the newly created N- and/or C-termini. Substitution mutations may also be performed, for example to stabilize the newly created termini or linker region.

It is also possible to modify the linker sequence. For example, free cysteine residues in the linker sequence may be replaced with less reactive residues, or large hydrophobic residues may be replaced with alternate residues that are less likely to promote aggregation.

In a preferred embodiment, the immunogenicity of interferons may be modulated. See for example U.S. Ser. Nos: 09/903,378; 10/039,170; 10/339,788 (filed Jan. 8, 2003, titled Novel Protein with Altered Immunogenicity); and PCT/US01/21823; and PCT/US02/00165. All references expressly incorporated by reference in their entirety.

In an alternate preferred embodiment, the interferon variant is further modified to increase stability. For example by decreasing the concentration of partially unfolded, aggregation-prone species. For example, modifications can be introduced to the protein core that improve packing or remove polar or charged groups that are not forming favorable hydrogen bond or electrostatic interactions. It is also possible to introduce modifications that introduce stabilizing electrostatic interactions or remove destabilizing interactions.

In one embodiment, the sequence of the variant interferon protein is modified in order to add or remove one or more N-linked or O-linked glycosylation sites. Addition of glycosylation sites to variant interferon polypeptides may be accomplished, for example, by the incorporation of one or more serine or threonine residues to the native sequence or variant interferon polypeptide (for O-linked glycosylation sites) or by the incorporation of a canonical N-linked glycosylation site, N-X-Y, where X is any amino acid except for proline and Y is threonine, serine or cysteine. Glycosylation sites may be removed by replacing one or more serine or threonine residues or by replacing one or more N-linked glycosylation sites.

In another preferred embodiment, one or more cysteine, lysine, histidine, or other reactive amino acids are designed into variant interferon proteins in order to incorporate labeling sites or PEGylation sites. It is also possible to remove one or more cysteine, lysine, histidine, or other reactive amino acids in order to prevent the incorporation of labeling sites or PEGylations sites at specific locations. For example, in a preferred embodiment, non-labile PEGylation sites are selected to be well removed from any required receptor binding sites in order to minimize loss of activity.

Variant interferon polypeptides of the present invention may also be modified to form chimeric molecules comprising a variant interferon polypeptide fused to another, heterologous polypeptide or amino acid sequence. In one embodiment, such a chimeric molecule comprises a fusion of a variant interferon polypeptide with a tag polypeptide which provides an epitope to which an anti-tag antibody can selectively bind. The epitope tag is generally placed at the amino- or carboxyl-terminus of the variant interferon polypeptide. The presence of such epitope-tagged forms of a variant interferon polypeptide can be detected using an antibody against the tag polypeptide. Also, provision of the epitope tag enables the variant interferon polypeptide to be readily purified by affinity purification using an anti-tag antibody or another type of affinity matrix that binds to the epitope tag. Various tag polypeptides and their respective antibodies are well known in the art. Examples include poly-histidine (poly-His) or poly-histidine-glycine (poly-His-Gly) tags; the flu HA tag polypeptide and its antibody 12CA5 [Field et al., Mol. Cell. Biol. 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6): 547-553 (1990)]. Other tag polypeptides include the Flag-peptide [Hopp et al., BioTechnology 6:1204-1210 (1988)]; the KT3 epitope peptide [Martin et al., Science 255:192-194 (1992)]; tubulin epitope peptide [Skinner et al., J. Biol. Chem. 266:15163-15166 (1991)]; and the T7 gene 10 protein peptide tag [Lutz-Freyermuth et al., Proc. Natl. Acad. Sci. U.S.A. 87:6393-6397 (1990)].

In an alternative embodiment, the chimeric molecule may comprise a fusion of a variant interferon polypeptide with another protein. Various fusion partners are well known in the art, and include but are not limited to the following examples. The variant interferon proteins of the invention may be fused to an immunoglobulin or the Fc region of an immunoglobulin, such as an IgG molecule. The interferon variants can also be fused to albumin, other interferon proteins, other cytokine proteins, the extracellular domains of the interferon receptor protein, etc.

In an especially preferred embodiment, rational design of improved IFN variants is achieved by using Protein Design Automation® (PDA®) technology. (See U.S. Pat. Nos. 6,188,965; 6,269,312; 6,403,312; WO98/47089 and U.S. Ser. Nos. 09/058,459, 09/127,926, 60/104,612, 60/158,700, 09/419,351, 60/181,630, 60/186,904, 09/419,351, 09/782,004 and 09/927,790, 60/347,772, and 10/218,102; and PCT/US01/218,102 and U.S. Ser. No. 10/218,102, U.S. S. No. 60/345,805; U.S. S. No. 60/373,453 and U.S. S. No. 60/374,035, all references expressly incorporated herein in their entirety.)

PDA® technology couples computational design algorithms that generate quality sequence diversity with experimental high-throughput screening to discover proteins with improved properties. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions.

In a preferred embodiment, each polar residue is represented using a set of discrete low-energy side-chain conformations (see for example Dunbrack Curr. Opin. Struct. Biol. 12:431-440 (2002). A preferred force field may include terms describing van der Waals interactions, hydrogen bonds, electrostatic interactions, and solvation, among others.

In a preferred embodiment, Dead-End Elimination (DEE) is used to identify the rotamer for each polar residue that has the most favorable energy (see Gordon et. al. J. Comput Chem. 24: 232-243 (2003), Goldstein Biophys. J. 66: 1335-1340 (1994) and Lasters and Desmet, Prot. Eng. 6: 717-722 (1993)).

In an alternate embodiment, Monte Carlo can be used in conjunction with DEE to identify groups of polar residues that have favorable energies.

In a preferred embodiment, after performing one or more PDA® technology calculations, a library of variant proteins is designed, experimentally constructed, and screened for desired properties.

In an alternate preferred embodiment, a sequence prediction algorithm (SPA) is used to design proteins that are compatible with a known protein backbone structure as is described in Raha, K., et al. (2000) Protein Sci., 9: 1106-1119; U.S. Ser. No. 09/877,695, filed Jun. 8, 2001 and Ser. No. 10/071,859, filed Feb. 6, 2002.

In one embodiment, the library is a combinatorial library, meaning that the library comprises all possible combinations of allowed residues at each of the variable positions.

Generating the Variants

Variant interferon nucleic acids and proteins of the invention may be produced using a number of methods known in the art.

Preparing Nucleic Acids Encoding the IFN Variants

In a preferred embodiment, nucleic acids encoding IFN variants are prepared by total gene synthesis, or by site-directed mutagenesis of a nucleic acid encoding wild type or variant IFN protein. Methods including template-directed ligation, recursive PCR, cassette mutagenesis, site-directed mutagenesis or other techniques that are well known in the art may be utilized (see for example Strizhov et. al. PNAS 93:15012-15017 (1996), Prodromou and Perl, Prot. Eng. 5: 827-829 (1992), Jayaraman and Puccini, Biotechniques 12: 392-398 (1992), and Chalmers et. at. Biotechniques 30: 249-252 (2001)).

Expression Vectors

In a preferred embodiment, an expression vector that comprises the components described below and a gene encoding a variant IFN protein is prepared. Numerous types of appropriate expression vectors and suitable regulatory sequences for a variety of host cells are known in the art. The expression vectors may contain transcriptional and translational regulatory sequences including but not limited to promoter sequences, ribosomal binding sites, transcriptional start and stop sequences, translational start and stop sequences, transcription terminator signals, polyadenylation signals, and enhancer or activator sequences. In a preferred embodiment, the regulatory sequences include a promoter and transcriptional start and stop sequences. In addition, the expression vector may comprise additional elements. For example, the expression vector may have two replication systems, thus allowing it to be maintained in two organisms, for example in mammalian or insect cells for expression and in a prokaryotic host for cloning and amplification. Furthermore, for integrating expression vectors, the expression vector contains at least one sequence homologous to the host cell genome, and preferably two homologous sequences, which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used. The expression vectors may be either self-replicating extrachromosomal vectors or vectors which integrate into a host genome.

The expression vector may include a secretory leader sequence or signal peptide sequence that provides for secretion of the variant IFN protein from the host cell. Suitable secretory leader sequences that lead to the secretion of a protein are known in the art. The signal sequence typically encodes a signal peptide comprised of hydrophobic amino acids, which direct the secretion of the protein from the cell. The protein is either secreted into the growth media or, for prokaryotes, into the periplasmic space, located between the inner and outer membrane of the cell. For expression in bacteria, bacterial secretory leader sequences, operably linked to a variant IFN encoding nucleic acid, are usually preferred.

Transfection/Transformation

The variant IFN nucleic acids are introduced into the cells either alone or in combination with an expression vector in a manner suitable for subsequent expression of the nucleic acid. The method of introduction is largely dictated by the targeted cell type. Exemplary methods include CaPO₄ precipitation, liposome fusion, Lipofectin®, electroporation, viral infection, dextran-mediated transfection, polybrene mediated transfection, protoplast fusion, direct microinjection, etc. The variant IFN nucleic acids may stably integrate into the genome of the host cell or may exist either transiently or stably in the cytoplasm. As outlined herein, a particularly preferred method utilizes retroviral infection, as outlined in PCT/US97/01019, incorporated by reference.

Hosts for the Expression of IFN Variants

Appropriate host cells for the expression of IFN variants include yeast, bacteria, archaebacteria, fungi, and insect and animal cells, including mammalian cells. Of particular interest are bacteria such as E. coli and Bacillus subtilis, fungi such as Saccharomyces cerevisiae, Pichia pastoris, and Neurospora, insects such as Drosophila melangaster and insect cell lines such as SF9, mammalian cell lines including 293, CHO, COS, Jurkat, NIH3T3, etc (see the ATCC cell line catalog, hereby expressly incorporated by reference), as well as primary cell lines.

Interferon variants can also be produced in more complex organisms, including but not limited to plants (such as corn, tobacco, and algae) and animals (such as chickens, goats, cows); see for example Dove, Nature Biotechnol. 20: 777-779 (2002).

In one embodiment, the cells may be additionally genetically engineered, that is, contain exogenous nucleic acid other than the expression vector comprising the variant IFN nucleic acid.

Expression Methods

The variant IFN proteins of the present invention are produced by culturing a host cell transformed with an expression vector containing nucleic acid encoding a variant IFN protein, under the appropriate conditions to induce or cause expression of the variant IFN protein. The conditions appropriate for variant IFN protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Purification

In a preferred embodiment, the IFN variants are purified or isolated after expression. Standard purification methods include electrophoretic, molecular, immunological and chromatographic techniques, including ion exchange, hydrophobic, affinity, and reverse-phase HPLC chromatography, and chromatofocusing. For example, a IFN variant may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3d ed. (1994). The degree of purification necessary will vary depending on the desired use, and in some instances no purification will be necessary. For further references on purification of type I interferons, see for example Moschera et al. Meth. Enzym. 119: 177-183 (1986); Tarnowski et al. Meth. Enzym. 119:153-165(1986); Thatcher et al. Meth. Enzym. 119:166-177 (1986); Lin et al. Meth. Enzym. 119:183-192 (1986). Methods for purification of interferon beta are disclosed in U.S. Pat. No. 4,462,940 and U.S. Pat. No. 4,894,330.

Posttranslational Modification and Derivitization

Once made, the variant IFN proteins may be covalently modified. Covalent and non-covalent modifications of the protein are thus included within the scope of the present invention. Such modifications may be introduced into a variant IFN polypeptide by reacting targeted amino acid residues of the polypeptide with an organic derivatizing agent that is capable of reacting with selected side chains or terminal residues. Optimal sites for modification can be chosen using a variety of criteria, including but not limited to, visual inspection, structural analysis, sequence analysis and molecular simulation.

In one embodiment, the variant IFN proteins of the invention are labeled with at least one element, isotope or chemical compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position. Labels include but are not limited to biotin, tag (e.g. FLAG, Myc) and fluorescent labels (e.g. fluorescein).

Derivatization with bifunctional agents is useful, for instance, for cross linking a variant IFN protein to a water-insoluble support matrix or surface for use in the method for purifying anti-variant IFN antibodies or screening assays, as is more fully described below. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio] propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the “—amino groups of lysine, arginine, and histidine side chains (T. E. Creighton, Proteins: Structure and Molecular Properties, W.H. Freeman & Co., San Francisco, pp. 79-86 (1983)), acetylation of the N-terminal amine, and amidation of any C-terminal carboxyl group.

Such derivitization may improve the solubility, absorption, permeability across the blood brain barrier, serum half life, and the like. Modifications of variant IFN polypeptides may alternatively eliminate or attenuate any possible undesirable side effect of the protein. Moieties capable of mediating such effects are disclosed, for example, in Remington's Pharmaceutical Sciences, 16th ed., Mack Publishing Co., Easton, Pa. (1980).

Another type of covalent modification of variant IFN comprises linking the variant IFN polypeptide to one of a variety of nonproteinaceous polymers, e.g., polyethylene glycol (“PEG”), polypropylene glycol, or polyoxyalkylenes, in the manner set forth in U.S. Pat. Nos. 4,640,835; 4,496,689; 4,301,144; 4,670,417; 4,791,192 or 4,179,337. A variety of coupling chemistries may be used to achieve PEG attachment, as is well known in the art. Examples, include but are not limited to, the technologies of Shearwater and Enzon, which allow modification at primary amines, including but not limited to, cysteine groups, histidine groups, lysine groups and the N-terminus (see, Kinstler et al, Advanced Drug Deliveries Reviews, 54, 477-485 (2002) and M J Roberts et al, Advanced Drug Delivery Reviews, 54, 459-476 (2002)). Both labile and non-labile PEG linkages may be used.

An additional form of covalent modification includes coupling of the variant IFN polypeptide with one or more molecules of a polymer comprised of a lipophililic and a hydrophilic moiety. Such composition may enhance resistance to hydrolytic or enzymatic degradation of the IFN protein. Polymers utilized may incorporate, for example, fatty acids for the lipophilic moiety and linear polyalkylene glycols for the hydrophilic moiety. The polymers may additionally incorporate acceptable sugar moieties as well as spacers used for IFN protein attachment. Polymer compositions and methods for covalent conjugation are described, for example, in U.S. Pat. Nos. 5,681,811; 5,359,030.

Another type of modification is chemical or enzymatic coupling of glycosides to the variant IFN protein. Such methods are described in the art, e.g., in WO 87/05330 published 11 Sep. 1987, and in Aplin and Wriston, CRC Crit. Rev. Biochem., pp. 259-306 (1981).

Alternatively, removal of carbohydrate moieties present on the variant IFN polypeptide may be accomplished chemically or enzymatically. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Assaying the Variants

In a preferred embodiment, the wild-type and variant proteins are analyzed for biological activities and physico-chemical properties by suitable methods known in the art.

Assays for stability include but are not limited to thermal or chemical denaturation assays (which may be performed under varying solution conditions, such as high salt or low pH), gastric stability assays, protease susceptibility assays, and the like.

Assays for solubility include, but are not limited to, differential light scattering experiments, analytical ultracentrifugation, size exclusion chromatography, and the like. Solubility may also be tested by monitoring the concentration of protein that remains in solution as a function of time or exposure to stresses such as increased temperature. It is also possible to assay for soluble expression by any of a number of methods.

Assays for immunogenicity include, but are not limited to, the following. Ex vivo T cell activation may be detected by monitoring the production of certain cytokines or the uptake of tritiated thymidine following the exposure of the T cells to matched antigen presenting cells that have been challenged with a peptide or whole protein of interest one or more times. In the most preferred embodiment, interferon gamma production is monitored using Elispot assays (see Schmittel et al. J. Immunol. Meth., 24: 17-24 (2000)). Immunogenicity can also measured in transgenic mouse systems. For example, mice expressing fully or partially human class II MHC molecules may be used. In another alternate embodiment, immunogenicity is tested by administering the IFN variants to one or more animals, including rodents and primates, and monitoring for antibody formation.

Assays for interferon activity include but are not limited to activation of interferon-responsive genes, receptor binding assays, antiviral activity assays, cytopathic effect inhibition assays, antiproliferative assays, immunomodulatory assays, and assays that monitor the induction of MHC molecules, all described in Meager, J. Immunol. Meth., 261:21-36 (2002).

In a preferred embodiment, wild type and variant proteins will be analyzed for their ability to activate interferon-sensitive signal transduction pathways. One example is the interferon-stimulated response element (ISRE) assay. Cells which constitutively express the type I interferon receptor are transiently transfected with an ISRE-luciferase vector. After transfection, the cells are treated with an interferon variant. In a preferred embodiment, a number of protein concentrations, for example from 0.0001-10 ng/mL, are tested to generate a dose-response curve. In an alternate embodiment, two or more concentrations are tested. If the variant binds and activates its receptor, the resulting signal transduction cascade induces luciferase expression. Luminescence can be measured in a number of ways, for example by using a TopCount™ or Fusion™ microplate reader.

In a preferred embodiment, wild type and variant proteins will be analyzed for their ability to bind to the type I interferon receptor (IFNAR). Suitable binding assays include, but are not limited to, BIAcore assays (Pearce et al., Biochemistry 38:81-89 (1999)) and AlphaScreen™ assays (commercially available from PerkinElmer) (Bosse R., Illy C., and Chelsky D (2002). Principles of AlphaScreen™ PerkinElmer Literature Application Note Ref# s4069. AlphaScreen™ is a bead-based non-radioactive luminescent proximity assay where the donor beads are excited by a laser at 680 nm to release singlet oxygen. The singlet oxygen diffuses and reacts with the thioxene derivative on the surface of acceptor beads leading to fluorescence emission at ˜600 nm. The fluorescence emission occurs only when the donor and acceptor beads are brought into close proximity by molecular interactions occurring when each is linked to ligand and receptor respectively. This ligand-receptor interaction can be competed away using receptor-binding variants while non-binding variants will not compete.

In an alternate preferred embodiment, wild type and variant proteins will be analyzed for their efficacy in treating an animal model of disease, such as the mouse or rat EAE model for multiple sclerosis.

The cyclic and circularly permuted interferon variants may also be tested to determine whether they are suitable for alternative (i.e. non-injection based) modes of delivery. For example, a cyclic interferon with enhanced stability may be suitable for oral delivery.

Administration and Treatment Using IFN Variants

Once made, the variant IFN proteins and nucleic acids of the invention find use in a number of applications. In a preferred embodiment, a variant IFN protein or nucleic acid is administered to a patient to treat an IFN related disorder.

The administration of the variant IFN proteins of the present invention, preferably in the form of a sterile aqueous solution, may be done in a variety of ways, including, but not limited to, orally, parenterally, subcutaneously, intravenously, intranasally, transdermally, intraperitoneally, intramuscularly, intrapulmonary, vaginally, rectally, intranasally or intraocularly. In some instances, the variant IFN protein may be directly applied as a solution or spray. Depending upon the manner of introduction, the pharmaceutical composition may be formulated in a variety of ways.

The pharmaceutical compositions of the present invention comprise a variant IFN protein in a form suitable for administration to a patient. In the preferred embodiment, the pharmaceutical compositions are in a water-soluble form, such as being present as pharmaceutically acceptable salts, which is meant to include both acid and base addition salts.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations.

In a further embodiment, the variant IFN proteins are added in a micellular formulation; see U.S. Pat. No. 5,833,948.

Combinations of pharmaceutical compositions may be administered. Moreover, the compositions may be administered in combination with other therapeutics.

In a preferred embodiment, the nucleic acid encoding the variant IFN proteins may also be used in gene therapy. In gene therapy applications, genes are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. The oligonucleotides may be modified to enhance their uptake, e.g. by substituting their negatively charged phosphodiester groups by uncharged groups.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection (Dzau et al., Trends in Biotechnology 11:205-210 (1993)). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990). For review of gene marking and gene therapy protocols see Anderson et al., Science 256:808-813 (1992).

While the foregoing invention has been described above, it will be clear to one skilled in the art that various changes and additional embodiments made be made without departing from the scope of the invention. All publications, patents, patent applications (provisional, utility and PCT) or other documents cited herein are incorporated by references in their entirety.

We claim:

1. A type 1 interferon (IFN) comprising antiviral, antineoplastic or immunomodulatory activity similar to a naturally occurring interferon, wherein said IFN has been circularly permuted or cyclized and has at least one modulated characteristic as compared to the naturally occurring interferon.

2. An IFN according to claim 1, wherein said IFN is circularly permuted.

3. An IFN according to claim 2, wherein said IFN is selected from the group consisting of: IFN-alpha, IFN-beta, IFN-kappa, IFN-omega and IFN-tau.

4. An IFN according to claim 3, wherein said IFN is IFN-beta.

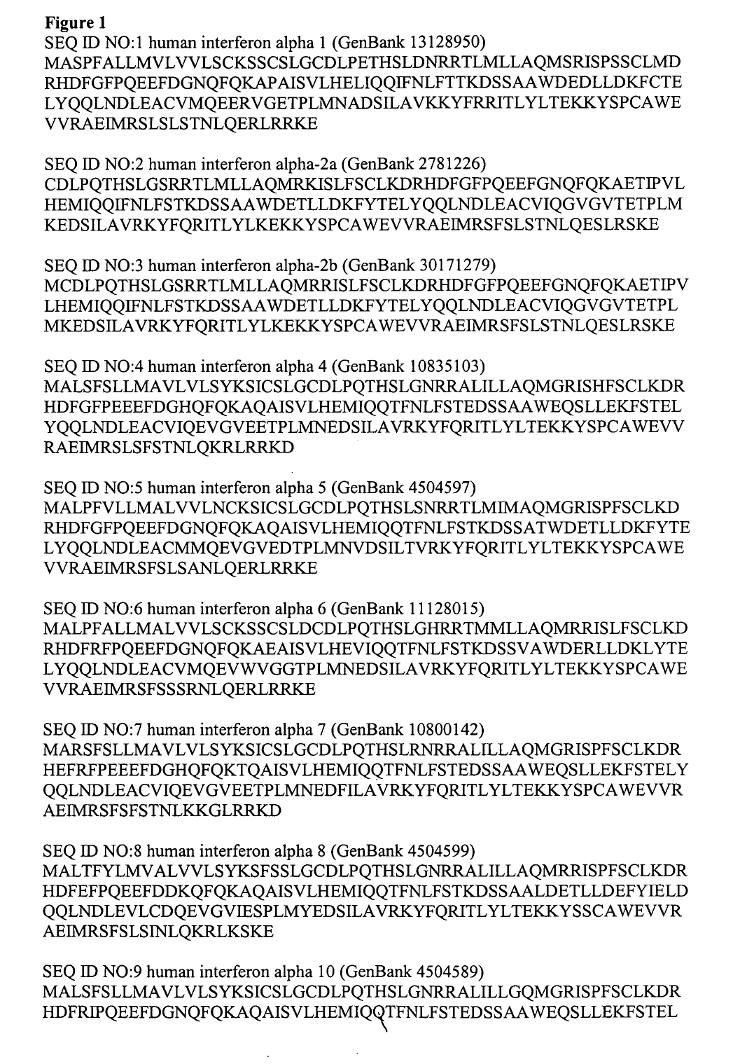

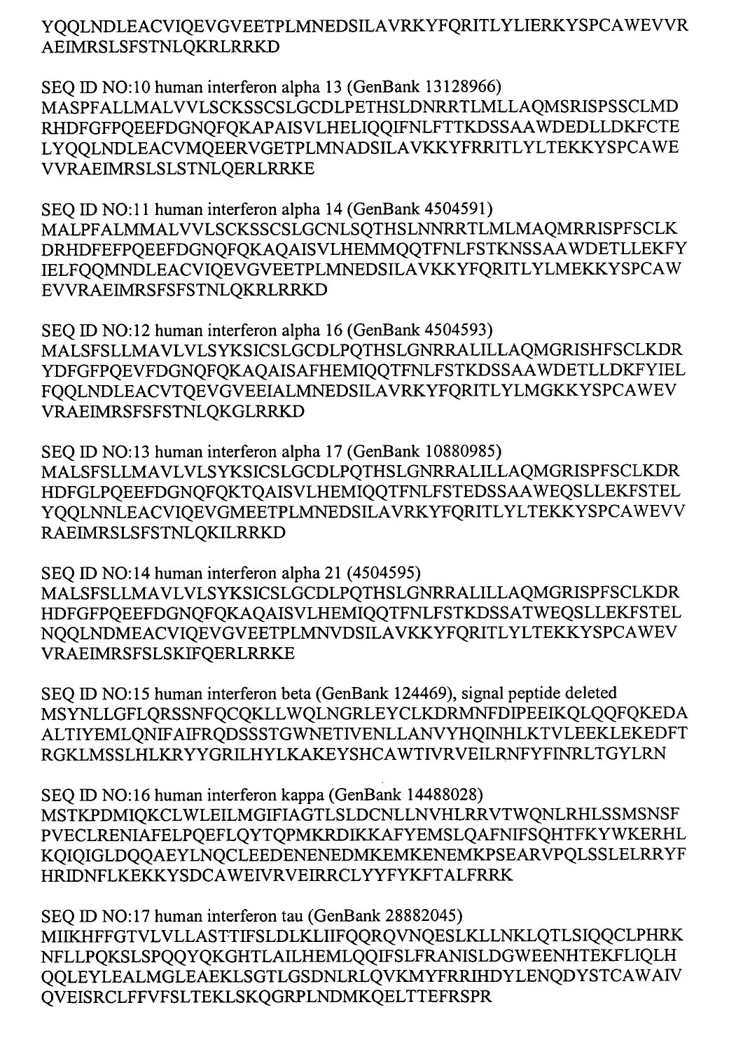

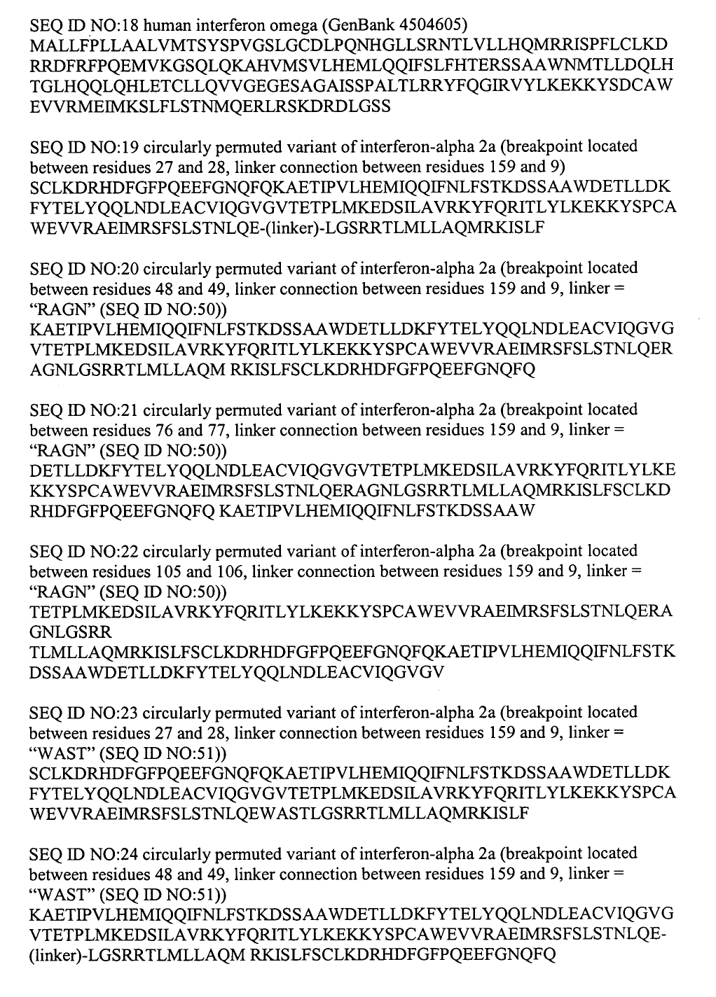

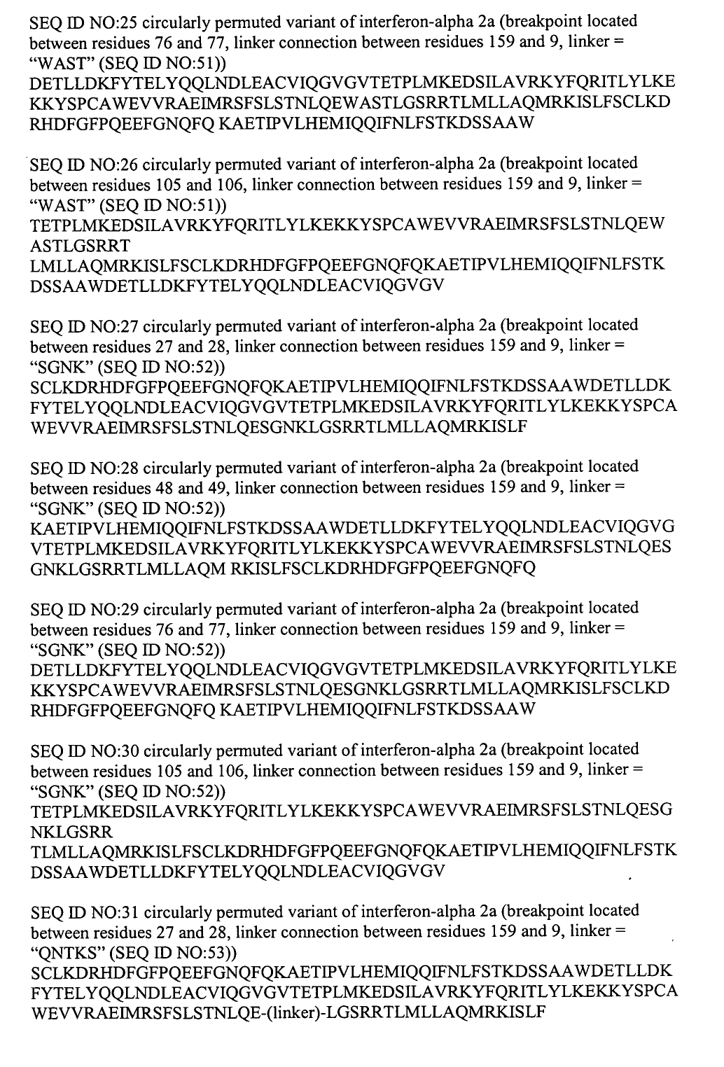

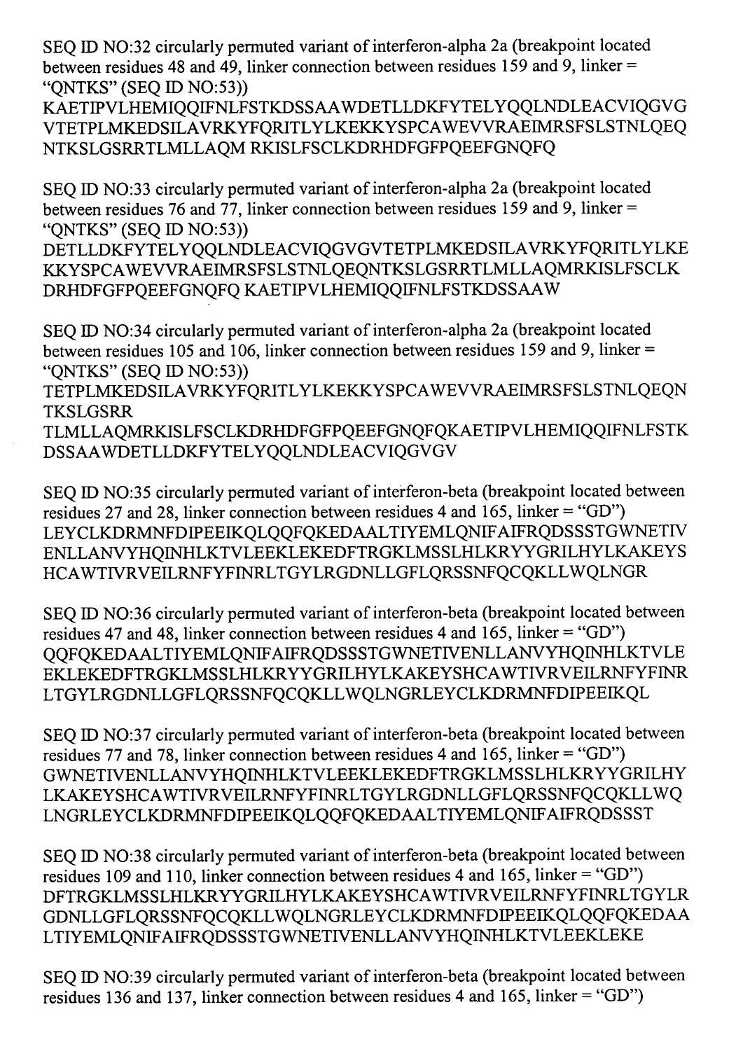

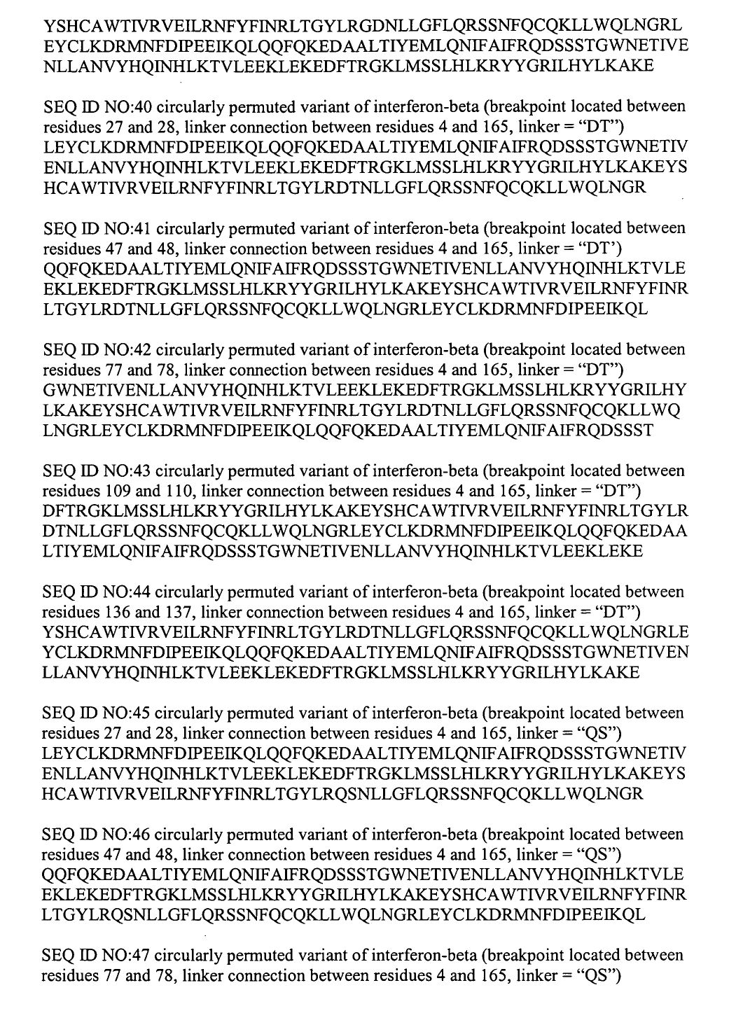

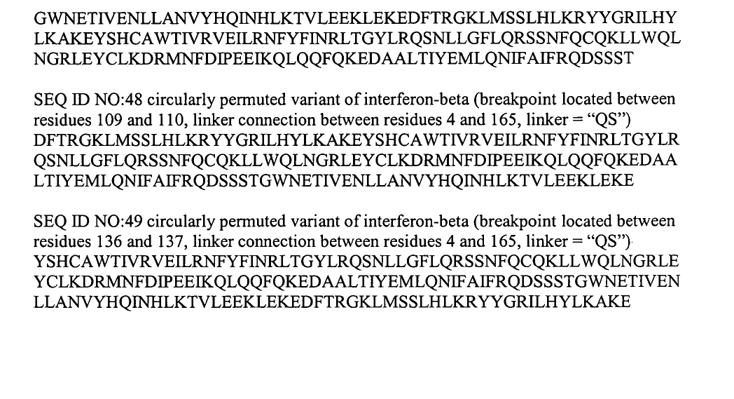

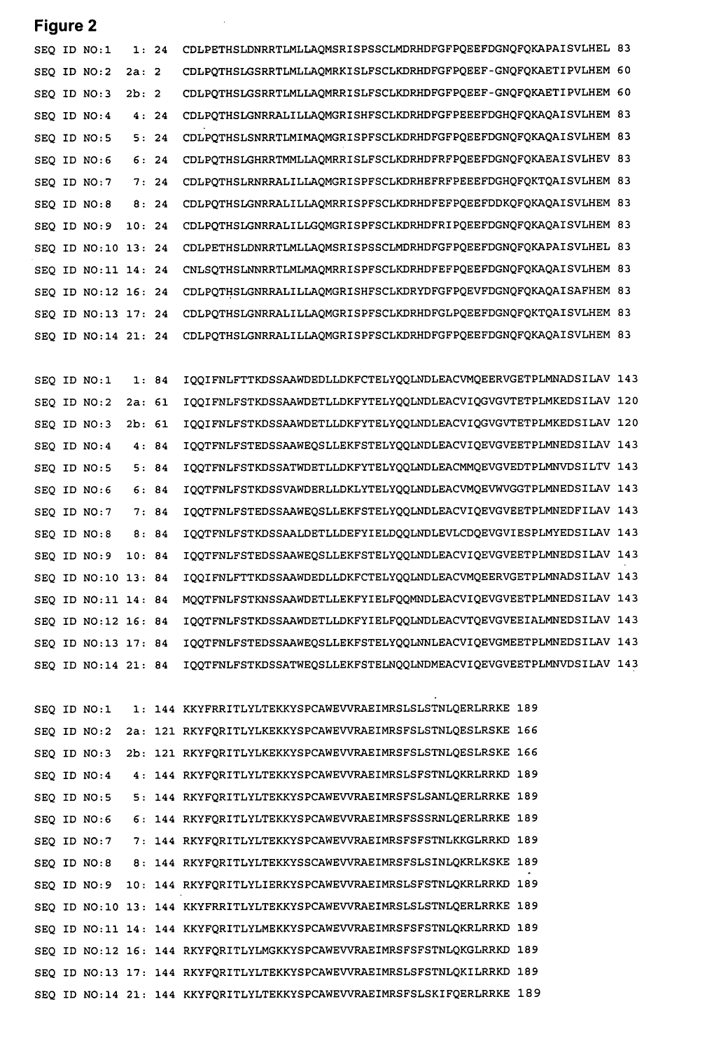

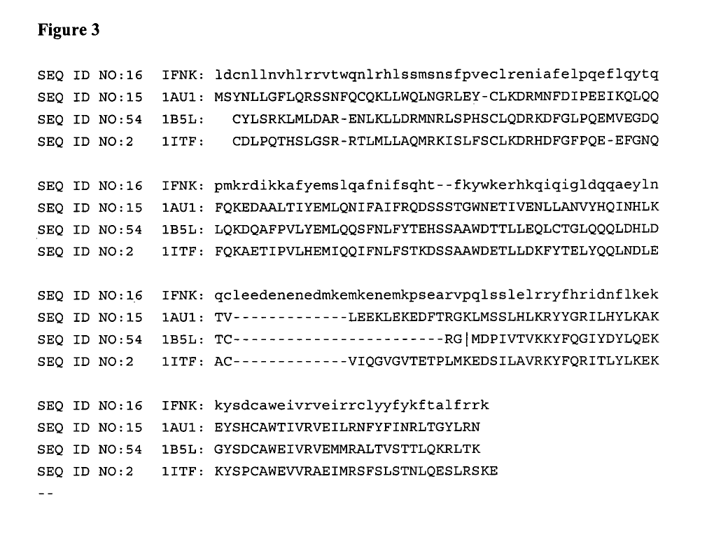

5. An IFN according to claim 4, wherein said circularly permuted interferon is selected from FIG. 1, SEQUENCE ID Nos. 35-49.

6. An IFN according to claim 3, wherein said IFN is IFN-alpha.

7. An IFN according to claim 5, wherein said circularly permuted interferon is selected from FIG. 1SEQUENCE ID Nos. 19-34.

8. An IFN according to claim 2, wherein said modulated characteristic is selected from the group consisting of: stability, solubility, activity, pharmakokinetics and immunogenicity.

9. An IFN according to claim 6, wherein said modulated characteristic is designed using a protein design computational program to achieve said characteristic.

10. An IFN according to claim 7, wherein said protein design computational program is PDA®).

11. An IFN according to claim 1, wherein said IFN is further chemically modified.

12. An IFN according to claim 9, wherein said chemical modification is glycosylation or PEGylation.

13. A recombinant nucleic acid encoding an IFN of claim 1.

14. An expression vector comprising the recombinant nucleic acid of claim 13.

15. A host cell comprising the recombinant nucleic acid of claim 13.

16. A host cell comprising the expression vector of claim 14.

17. A method of producing an IFN comprising culturing the host cell of claim 16 under conditions suitable for expression of said nucleic acid.

18. The method according to claim 17 further comprising recovering said IFN.

19. An IFN composition comprising a pharmaceutically acceptable carrier and an IFN of claim 1.


SEQUENCE LISTING (54 sequences)

SEQ ID NO: 1 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 2 - protein, 165 aa, Homo sapiens
CDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 3 - protein, 166 aa, Homo sapiens [sequence omitted]

SEQ ID NO: 4 - protein, 189 aa, Homo sapiens
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 5 - protein, 189 aa, Homo sapiens
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 6 - protein, 189 aa, Homo sapiens
MALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 7 - protein, 189 aa, Homo sapiens
MARSFSLLMAVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLTEKKYSPCAWEVVRAEIMRSFSFSTNLKKGLRRKD

SEQ ID NO: 8 - protein, 189 aa, Homo sapiens
MALTFYLMVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLEVLCDQEVGVIESPLMYEDSILAVRKYFQRITLYLTEKKYSSCAWEVVRAEIMRSFSLSINLQKRLKSKE

SEQ ID NO: 9 - protein, 189 aa, Homo sapiens
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 10 - protein, 189 aa, Homo sapiens
MASPFALLMALVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 11 - protein, 189 aa, Homo sapiens
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSFSFSTNLQKRLRRKD

SEQ ID NO: 12 - protein, 189 aa, Homo sapiens
MALSFSLLMAVLVLSYKSICSLGCDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 13 - protein, 189 aa, Homo sapiens
MALSFSLLMAVLVLSYKSICSLGCDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 14 - protein, 189 aa, Homo sapiens
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDMEACVIQEVGVEETPLMNVDSILAVKKYFQRITLYLTEKKYSPCAWEVVRAEIMRSFSLSKIFQERLRRKE

SEQ ID NO: 15 - protein, 166 aa, Homo sapiens
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 16 - protein, 207 aa, Homo sapiens
MSTKPDMIQKCLWLEILMGIFIAGTLSLDCNLLNVHLRRVTWQNLRHLSSMSNSFPVECLRENIAFELPQEFLQYTQPMKRDIKKAFYEMSLQAFNIFSQHTFKYWKERHLKQIQIGLDQQAEYLNQCLEEDENENEDMKEMKENEMKPSEARVPQLSSLELRRYFHRIDNFLKEKKYSDCAWEIVRVEIRRCLYYFYKFTALFRRK

SEQ ID NO: 17 - protein, 208 aa, Homo sapiens
MIIKHFFGTVLVLLASTTIFSLDLKLIIFQQRQVNQESLKLLNKLQTLSIQQCLPHRKNFLLPQKSLSPQQYQKGHTLAILHEMLQQIFSLFRANISLDGWEENHTEKFLIQLHQQLEYLEALMGLEAEKLSGTLGSDNLRLQVKMYFRRIHDYLENQDYSTCAWAIVQVEISRCLFFVFSLTEKLSKQGRPLNDMKQELTTEFRSPR

SEQ ID NO: 18 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 19 - protein, 151 aa, Artificial
  synthetic
SCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQELGSRRTLMLLAQMRKISLF

SEQ ID NO: 20 - protein, 155 aa, Artificial
  synthetic
KAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQERAGNLGSRRTLMLLAQMRKISLFSCLKDRHDFGFPQEEFGNQFQ

SEQ ID NO: 21 - protein, 155 aa, Artificial
  synthetic
DETLLDKFYTELYQQLNDLEACVIQGVGVTETPLMKEDSILAVRKYFQRITLYLKEKKYSPCAWEVVRAEIMRSFSLSTNLQERAGNLGSRRTLMLLAQMRKISLFSCLKDRHDFGFPQEEFGNQFQKAETIPVLHEMIQQIFNLFSTKDSSAAW

SEQ ID NO: 22 - protein, 155 aa, Artificial
  synthetic
TETPLMKEDSILAVRKYFQRITLYLKEKKYSPCAWEVVRAEIMRSFSLSTNLQERAGNLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGV

SEQ ID NO: 23 - protein, 155 aa, Artificial
  synthetic
SCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEWASTLGSRRTLMLLAQMRKISLF

SEQ ID NO: 24 - protein, 155 aa, Artificial
  synthetic
KAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEWASTLGSRRTLMLLAQMRKISLFSCLKDRHDFGFPQEEFGNQFQ

SEQ ID NO: 25 - protein, 155 aa, Artificial
  synthetic
DETLLDKFYTELYQQLNDLEACVIQGVGVTETPLMKEDSILAVRKYFQRITLYLKEKKYSPCAWEVVRAEIMRSFSLSTNLQEWASTLGSRRTLMLLAQMRKISLFSCLKDRHDFGFPQEEFGNQFQKAETIPVLHEMIQQIFNLFSTKDSSAAW

SEQ ID NO: 26 - protein, 155 aa, Artificial
  synthetic
TETPLMKEDSILAVRKYFQRITLYLKEKKYSPCAWEVVRAEIMRSFSLSTNLQEWASTLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGV

SEQ ID NO: 27 - protein, 155 aa, Artificial
  synthetic
SCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESGNKLGSRRTLMLLAQMRKISLF

SEQ ID NO: 28 - protein, 155 aa, Artificial
  synthetic
KAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESGNKLGSRRTLMLLAQMRKISLFSCLKDRHDFGFPQEEFGNQFQ

SEQ ID NO: 29 - protein, 155 aa, Artificial
  synthetic
DETLLDKFYTELYQQLNDLEACVIQGVGVTETPLMKEDSILAVRKYFQRITLYLKEKKYSPCAWEVVRAEIMRSFSLSTNLQESGNKLGSRRTLMLLAQMRKISLFSCLKDRHDFGFPQEEFGNQFQKAETIPVLHEMIQQIFNLFSTKDSSAAW

SEQ ID NO: 30 - protein, 155 aa, Artificial
  synthetic
TETPLMKEDSILAVRKYFQRITLYLKEKKYSPCAWEVVRAEIMRSFSLSTNLQESGNKLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGV

SEQ ID NO: 31 - protein, 156 aa, Artificial
  synthetic
SCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEQNTKSLGSRRTLMLLAQMRKISLF

SEQ ID NO: 32 - protein, 156 aa, Artificial
  synthetic
KAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQEQNTKSLGSRRTLMLLAQMRKISLFSCLKDRHDFGFPQEEFGNQFQ

SEQ ID NO: 33 - protein, 156 aa, Artificial
  synthetic
DETLLDKFYTELYQQLNDLEACVIQGVGVTETPLMKEDSILAVRKYFQRITLYLKEKKYSPCAWEVVRAEIMRSFSLSTNLQEQNTKSLGSRRTLMLLAQMRKISLFSCLKDRHDFGFPQEEFGNQFQKAETIPVLHEMIQQIFNLFSTKDSSAAW

SEQ ID NO: 34 - protein, 156 aa, Artificial
  synthetic
TETPLMKEDSILAVRKYFQRITLYLKEKKYSPCAWEVVRAEIMRSFSLSTNLQEQNTKSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGV

SEQ ID NO: 35 - protein, 164 aa, Artificial
  synthetic
LEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRGDNLLGFLQRSSNFQCQKLLWQLNGR

SEQ ID NO: 36 - protein, 164 aa, Artificial
  synthetic
QQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRGDNLLGFLQRSSNFQCQKLLWQLNGRLEYCLKDRMNFDIPEEIKQL

SEQ ID NO: 37 - protein, 164 aa, Artificial
  synthetic
GWNETIVENLLANVYHQINHLKTVLEEKLEKEDFTRGKLMSSLHLKRYYGRILHYLKAKEYSHCAWTIVRVEILRNFYFINRLTGYLRGDNLLGFLQRSSNFQCQKLLWQLNGRLEYCLKDRMNFDIPEEIKQLQQFQKEDAALTIYEMLQNIFAIFRQDSSST

SEQ ID NO: 38 - protein, 164 aa, Artificial
  synthetic
DFTRGKLMSSLHLKRYYGRILHYLKAKEYSHCAWTIVRVEILRNFYFINRLTGYLRGDNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKE

SEQ ID NO: 39 - protein, 164 aa, Artificial
  synthetic
YSHCAWTIVRVEILRNFYFINRLTGYLRGDNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKE

SEQ ID NO: 40 - protein, 164 aa, Artificial
  synthetic
LEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRDTNLLGFLQRSSNFQCQKLLWQLNGR

SEQ ID NO: 41 - protein, 164 aa, Artificial
  synthetic
QQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRDTNLLGFLQRSSNFQCQKLLWQLNGRLEYCLKDRMNFDIPEEIKQL

SEQ ID NO: 42 - protein, 164 aa, Artificial
  synthetic
GWNETIVENLLANVYHQINHLKTVLEEKLEKEDFTRGKLMSSLHLKRYYGRILHYLKAKEYSHCAWTIVRVEILRNFYFINRLTGYLRDTNLLGFLQRSSNFQCQKLLWQLNGRLEYCLKDRMNFDIPEEIKQLQQFQKEDAALTIYEMLQNIFAIFRQDSSST

SEQ ID NO: 43 - protein, 164 aa, Artificial
  synthetic
DFTRGKLMSSLHLKRYYGRILHYLKAKEYSHCAWTIVRVEILRNFYFINRLTGYLRDTNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKE

SEQ ID NO: 44 - protein, 164 aa, Artificial
  synthetic
YSHCAWTIVRVEILRNFYFINRLTGYLRDTNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKE

SEQ ID NO: 45 - protein, 164 aa, Artificial
  synthetic
LEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRQSNLLGFLQRSSNFQCQKLLWQLNGR

SEQ ID NO: 46 - protein, 164 aa, Artificial
  synthetic
QQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRQSNLLGFLQRSSNFQCQKLLWQLNGRLEYCLKDRMNFDIPEEIKQL

SEQ ID NO: 47 - protein, 164 aa, Artificial
  synthetic
GWNETIVENLLANVYHQINHLKTVLEEKLEKEDFTRGKLMSSLHLKRYYGRILHYLKAKEYSHCAWTIVRVEILRNFYFINRLTGYLRQSNLLGFLQRSSNFQCQKLLWQLNGRLEYCLKDRMNFDIPEEIKQLQQFQKEDAALTIYEMLQNIFAIFRQDSSST

SEQ ID NO: 48 - protein, 164 aa, Artificial
  synthetic
DFTRGKLMSSLHLKRYYGRILHYLKAKEYSHCAWTIVRVEILRNFYFINRLTGYLRQSNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKE

SEQ ID NO: 49 - protein, 164 aa, Artificial
  synthetic
YSHCAWTIVRVEILRNFYFINRLTGYLRQSNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKE

SEQ ID NO: 50 - protein, 4 aa, Artificial
  synthetic
RAGN

SEQ ID NO: 51 - protein, 4 aa, Artificial
  synthetic
WAST

SEQ ID NO: 52 - protein, 4 aa, Artificial
  synthetic
SGNK

SEQ ID NO: 53 - protein, 5 aa, Artificial
  synthetic
QNTKS

SEQ ID NO: 54 - protein, 152 aa, Homo sapiens
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGMDPIVTVKKYFQGIYDYLQEKGYSDCAWEIVRVEMMRALTVSTTLQKRLTK